(19) United States
(12) Patent Application Publication
(10) Pub. No.: US 2022/0192642 A1
(43) Pub. Date: Jun. 23, 2022

(54) BODILY SAMPLE COLLECTION DEVICE

(71) Applicant: CALLA LILY PERSONAL CARE LTD, London (GB)

(72) Inventors: Yu Sing (Alex) HOOI, London (GB); Vinh-Thang VO-TA, London (GB)

(21) Appl. No.: 17/601,223

(22) PCT Filed: Apr. 3, 2020
(86) PCT No.: PCT/GB2020/050893

§ 371 Date: Oct. 4, 2021

(30) Foreign Application Priority Data
Apr. 5, 2019 (GB) 1904853.7

(51) Int. Cl. A61B 10/00

(52) U.S. Cl. CPC A61B 10/0038; A61B 10/0051; A61B 2010/0074; A61B 10/007; A61B 10/0096

(57) ABSTRACT

A device for the collection of a bodily sample from an anatomical cavity, the device includes an impermeable sheath arranged and configured to receive a user's finger, the sheath comprising a distal end and a proximal end; an absorbent body positioned at the distal end of the sheath; and a hermetically closable pad positioned at the proximal end of the sheath.

The present invention relates to a sample collection device, more particularly, the present invention relates to a device for collecting sample from an anatomical cavity, such as the vaginal or rectal cavity.

Swab tests involve taking a sample of secretions with a device resembling a cotton bud, with a cotton swab attached to a rod. A well-known use of such a device is for the collection of saliva from the mouth of a user for DNA testing. The swab is rubbed against the mouth cavity, placed in a sterile tube or bag, and sent to a laboratory for testing. However, it is recommended to wear a glove in order to avoid contamination during the sample collection.

Vaginal swab tests have a significant role in the diagnosis of fungal and bacterial infections, and sexually and non-sexually transmitted diseases, because such conditions are often accompanied by unusual (e.g. excessive, abnormally coloured, malodorous) discharges and/or bleeding.

Self-testing kits are available and include a rigid swab device similar to those used in buccal swab tests. However, self-positioning of the device in the vaginal cavity can be difficult. The general recommendation is to be in a comfortable position, for example the position one would use to insert a tampon. Whilst the insertion of a soft cotton tampon is forgiving, the blind insertion of a long thin rigid stick is less so, so that vaginal swab collections often cause scratches, injury and/or bleeding, which can result in infection and inflammation. The insertion of the same type of swab device in the rectal cavity (for rectal swab testing) is even more delicate and better achieved by a medical professional. However, it is still the case that some patients prefer to carry out these sensitive collections themselves.

It is an object of the invention to at least alleviate the above-mentioned disadvantages, and to provide an alternative to existing products.

According to a first aspect of the invention, there is provided a device for the collection of a bodily sample from an anatomical cavity, the device comprising an impermeable sheath arranged and configured to receive a user's finger, said sheath comprising a distal end and a proximal end; an absorbent body positioned at the distal end of the sheath; and a hermetically closable pad positioned at the proximal end of the sheath.

The device according to the present invention enables the collection of a bodily sample from an anatomical cavity, without contamination of the sample. The user's finger is enclosed in the sheath so that there is no contact with the absorbent body, the anatomical cavity or the patient. The pad can be hermetically closed after collection of the sample, which moots the need for a separate sterile, uncontaminated receptacle (such as a tube with conventional swab sticks).

The device also enables a hygienic collection of the sample, leaving the user's hand unsoiled. The user's finger is surrounded by the impermeable sheath so that the finger does not directly contact the walls of the anatomical cavity. In addition, the pad positioned at the proximal end of the sheath acts as a shield between the user's hand and the patient and prevents the hand from contacting the patient's body. There is therefore no need to use separate gloves to handle the device.

The device according to the present invention allows an atraumatic, injury-free collection of the sample because it does not involve the use of any sharp or rigid parts, such as a thin rigid rod in conventional swab sticks. The risk of bleeding (a common side effect of sample collection using a swab stick) is also decreased.

Sample collection using the device according to the present invention is also easier, in that the absorbent body is guided directly by the user's finger. In the case of a rigid swab stick, the whole stick needs to be tilted in the patient's cavity (vaginal or rectal) in order to ensure that the swab contacts the anatomical wall of the cavity. This can be a difficult manipulation for the user, and an uncomfortable manipulation for the patient, in particular when the sample is collected from the vaginal or rectal cavity. By contrast, the absorbent body of the present device can be presented and contacted to the anatomical cavity by slightly bending or tilting the user's finger.

There is more freedom of movement and manipulation, which renders this device ideal of self-collection.

The absorbent body at the distal end of the sheath, and the pad is positioned at the proximal end of the sheath. In the context of the present invention, the distal end is the end of the sheath first inserted in the anatomical cavity, also corresponding to the end of the sheath contacting the tip of the user's finger during the insertion step; and the proximal end is positioned at or adjacent the cavity orifice or outside the anatomical cavity during the collection step.

It is envisaged that the absorbent body is replaced with another sample collecting means. The alternative sample collecting means may be non-absorbent. For example, the absorbent body may be replaced with a scraping means, such as a scraping brush as used to collect samples in smear tests.

Preferably, the pad is positioned outside the anatomical cavity. It is not specifically intended to be inserted into the anatomical cavity.

Preferably, the pad comprises two surfaces; in use (e.g. during the device insertion/sample collection process), the first surface faces the orifice of anatomical cavity (from which the sample is to be removed) of the patient (from whom the sample is collected) and the second surface faces away from the orifice of the anatomical cavity. Alternatively, it can be said that the first surface faces the tip of the user's (the person who collects the sample, who can be the same or different from the patient) finger and the second surface faces the user's arm.

In a preferred embodiment, the pad comprises a first surface facing, in use, the cavity, said first surface comprising an adhesive layer. After the sample has been collected, the sheath is reversed so that the absorbent body is located inside the sheath. The adhesive layer is located on the first surface of the pad so as to hermetically close the sheath.

The integrated pad is particularly advantageous for the medical practitioner collecting a sample from the patient, but also for the patient self-collecting the sample for example in the surgery or hospital bathrooms. Using a conventional swab stick, there is a risk that bodily fluids leak onto the user's or patient's hand. If the user or patient uses a glove, then the stick is handled and placed into the provided tube and the tube is closed, with a soiled gloved hand. Thus the tube itself will also potentially be soiled. Alternatively, the user or patient must remove the soiled glove before placing the stick into the tube, whilst holding the tube, which creates the risk of dropping (and contaminating) the sample.

Preferably, the pad comprises one or more lines of weaknesses (more preferably on line of weakness) about which the pad can be folded so as to facilitate the folding, closing and seal of the sheath.

Other hermetical sealing means are considered, including but not limited to mechanical sealing means such as grip seal and zip lock. However, such mechanical sealing means tend to comprises parts which can injure the patient and/or are prone to contamination. Thus, the adhesive sealing means is generally preferred.

Preferably, the adhesive is a pressure-sensitive adhesive. In this embodiment, the user can fold the pad in two and press the adhesive layer against itself to hermetically seal the sheath.

Preferably, the adhesive is a reseal adhesive. It is advantageous to use a resealable adhesive layer for the user, in case the pad was improperly folded, but also for the technicians analysing the sample. The laboratory technician might need to carry out multiple analysis and/or measurements, in which case the sample would need to be divided into several aliquots. The absorbent body can be removed from the sheath, divided into aliquots and stored as separate aliquots. Alternatively, the technician can remove an aliquot, reseal the sheath for later removal of a further aliquot.

Preferably, the adhesive layer is covered by a removable film. The film protects the adhesive layer until the sheath is to be closed. Therefore, the adhesive layer is not contaminated through handling by the user or contact with the patient. The film preferably covers the entire surface of the adhesive layer. The film may comprise a tab extending beyond the surface of the adhesive layer to facilitate the removal of the film from the pad. Preferably, the removable film comprises a first surface facing, in use, the anatomical cavity from which the sample is to be removed and a second surface facing the adhesive layer of the pad.

In a preferred embodiment, the first surface of the removable film comprises an absorbent layer. The absorbent layer provides additional protection for the user during the sample collection process, by absorbing bodily fluid leaking from the anatomical cavity.

In a preferred embodiment, the removable film comprising an impermeable layer to prevent bodily fluids from seeping through the film, onto the adhesive layer of the pad.

The pad may be substantially circular, oval, rectangular or any other suitable shape. The pad can be a standard rectangular napkin or a small interlabial pad. The shape of the pad is not limited as long as the dimensions and shape are sufficient to prevent substantial direct contact between the user's hand and the patient and does not hinder the user's movements.

In a preferred embodiment, the absorbent body comprises or consists of an absorbent layer secured to the distal end or tip of the sheath. A layer of absorbent material facilitates the manipulation of the absorbent body. However, it is envisaged that the absorbent body comprises or consists of a sheet, pad, swab or a small tampon-type body. For ease of manipulation, a layer or sheet is preferred, especially as usually only a small amount of sample is required for analysis.

It is also important to secure said absorbent body to the sheath so that it does not become detached from the sheath, thereby increasing the risk of infection (e.g. toxic shock syndrome) or contamination of the sample. It is also advantageous for the absorbent body to comprise a non-shedding absorbent material.

The absorbent body may be modified or comprise an additive, depending on the type of analysis to be carried out on the sample. For example, the absorbent body may comprise a charcoal media, which is particularly useful in case of fungal or bacterial infection. In a preferred embodiment, further comprising a removal string coupled to the distal end of the sheath. After the sample has been collected, the collection device is removed by pulling the string. The sheath reverses itself (outside in) so that the absorbent body which was outside the sheath, is now located inside the sheath.

In a second aspect of the present invention, there is provided the use for collecting a bodily sample from an anatomical cavity, of a device as described above.

Preferably, the anatomical cavity in the vaginal cavity, the rectal cavity or the buccal cavity. It is also envisaged to use the device according to the present invention to collect samples outside an anatomical cavity, for example a urine sample (by urinating on the absorbent body) or a faecal sample.

In a third aspect of the present invention, there is provided a method for collecting a bodily sample from an anatomical cavity comprising the steps of collecting a sample from said anatomical cavity using a device as described above; and hermetically closing the proximal end of the sheath.

The invention will be further described with reference to the drawings and figures, in which

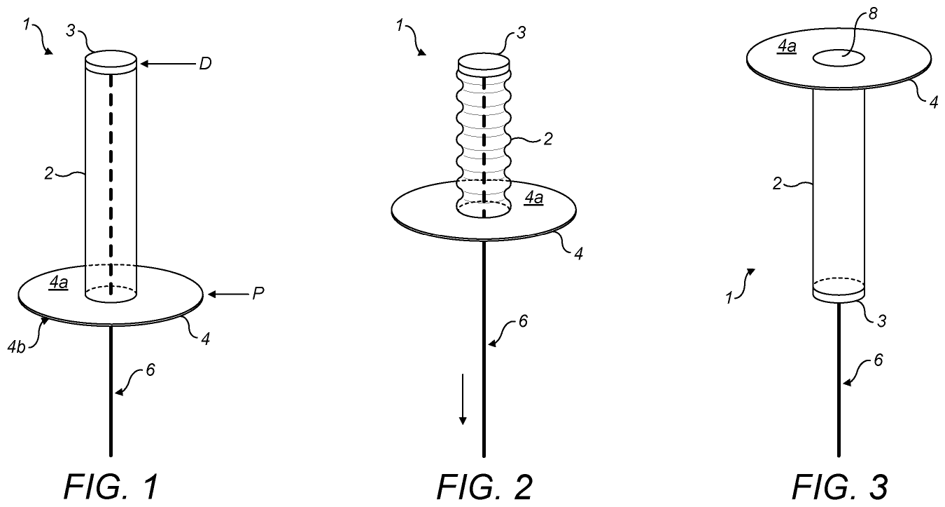

FIG. 1 is a schematic representation of a device according to the present invention in its insertion and collection configuration;

FIG. 2 is a schematic representation of the device of FIG. 1, being pulled out from the patient's cavity after collection of the sample;

FIG. 3 is a schematic representation of the device of FIG. 1 in its withdrawn configuration;

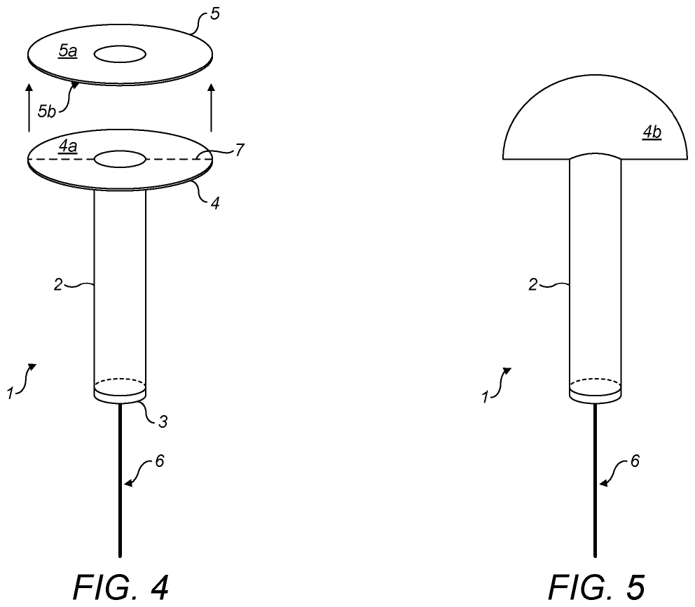

FIG. 4 is a schematic representation of the device of FIG. 1, with the cover being removed from the pad; and

FIG. 5 is a schematic representation of the device of FIG. 1, in its closed configuration.

Referring to FIG. 1, there is illustrated a device 1 for the collection of a bodily sample from an anatomical cavity, the device 1 comprising an impermeable sheath 2 arranged and configured to receive a user's finger (not shown), said sheath 2 comprising a distal end D and a proximal end P; an absorbent body 3 positioned at the distal end D of the sheath 1; and a hermetically closable pad 4 positioned at the proximal end P of the sheath 2.

The sheath 2 is substantially tubular. The sheath 2 comprises or consists of a material which is flexible at least in the radial direction so as to allow the user to insert a finger therein. The sheath 2 comprises a fluid impermeable material to prevent bodily fluids from soiling the user's finger.

The sheath 2 is preferably closed at its distal end to prevent bodily fluids absorbed by the absorbent body 3 from soiling or being contaminated by the user's finger. At its proximal end, the sheath 2 is opened to allow the user to insert his or her finger into the sheath 2. The opening at the proximal end of the sheath 2 substantially corresponds in dimensions and shape to an opening through the pad 4.

The pad 4 has a first surface 4a facing, in use, the patient's anatomical cavity and a second surface 4b which in use, faces away from the anatomical cavity. The first surface 4a comprises a layer of adhesive, which is preferably a pressure-sensitive adhesive. The layer of adhesive is covered by a removable film 5.

The second surface 4b of the pad 4 preferably comprises an impermeable layer. In a preferred embodiment, the sheath 2 extends through the aperture of the pad 4 and along the second surface 4b of the pad 4 to form an impermeable layer.

The pad 4 preferably comprises a line of weakness 7 about which the pad can be folded to hermetically close the sheath 2.

With reference to FIG. 4, the removable film 5 has a first surface 5a facing, in use, the patient's anatomical cavity and a second surface 5b which faces the layer of adhesive on the first surface 4a of the pad 4.

The dimensions and shape of the removable film 5 correspond to the dimensions and shape of the pad 4, but may comprise a tab (not shown) to facilitate the peeling away of the film 5 from the pad 4. The film 5 comprises an opening which substantially corresponds in dimensions and shape to the opening of the pad 4 and the sheath 2.

The first surface 5a of the film 5 may comprise an absorbent layer to absorb any fluid leaking from the anatomical cavity. The film 5 may comprise an impermeable layer, for example on its second surface 5b, to prevent contamination of the sample by the adhesive or of the adhesive by the sample.

An absorbent body 3 is secured to the distal end or tip of the sheath 2. In the device 1 shown in the figures, an absorbent sheet is secured to the distal end of the sheath 2, by bonding, gluing, sealing, sewing, welding or any other suitable securing method.

A string or cord 6 is coupled to the distal end of the sheath 2 to assist with the removal of the device 1 from the anatomical cavity.

In use, the device 1 is preferably packaged with the absorbent body 3 positioned inside the sheath 2 (see configuration of FIG. 3) so as to avoid any contamination before use. The user (i.e. the person collecting the sample) positions his or her finger at the distal end of the sheath 2, pushes the absorbent body 3 through the aperture 8 of the pad 4, and in the process, inserts his or her finger in the sheath 2. The absorbent body 3 is now exposed (see FIG. 1) and the device 1 is ready for insertion into the anatomical cavity of the patient.

The user inserts the sheathed finger through the anatomical orifice and into the anatomical cavity, for example, the vaginal cavity. Once the absorbent body 3 is at the target area, it can absorb any surrounding bodily fluid, or the user can contact the absorbent body 3 against the vaginal walls by bending his or her finger.

Once the sample has been collected, the absorbent body 3 is removed by pulling on the string 6. The sheath 2 reversed itself and the absorbent body 3 is now back inside the sheath 2 (see FIG. 3).

With reference to FIG. 4, the film 5 is peeled off the pad 4, thereby revealing the adhesive layer on the first surface 4a of the pad 4. The pad 4 is folded along the line of weakness 7 so that the adhesive layer contacts itself and pressure is applied on the second surface 4b of the pad 4 so as hermetically close the sheath 2. The sample is now contained within the sheath 2, which is hermetically closed and can be placed in a bag or enveloped for sending to the laboratory.

The technician can open the pad 4, insert his or her finger in the same manner so as to expose the sample. The sample can be removed by any suitable method. For example, the technician can directly place the sheathed finger in a suitable medium to extract, dilute or dissolve the sample from the absorbent body 3. If multiple analysis are to be carried out, the technician may remove an aliquot and reseal the sheath 2 for future extractions.

Thus, from the above description, it can be seen that the present invention provides a device and a method for the safe, sterile and hygienic collection of a sample from a patient's anatomical cavity. The device according to the present invention does not comprise any rigid or sharp parts, which could injure or hurt the patient so that the collection process is painless. The device is easier to manipulate than conventional swab stick so that it allows patients to opt of self-collection. The risk of contamination is minimised as there is no contact with the absorbent body by the user. The sample collection process also minimises the risk of soiling the user's or patient's fingers.

1. A device for the collection of a bodily sample from an anatomical cavity, the device comprising:
an impermeable sheath arranged and configured to receive a user's finger, said sheath comprising a distal end and a proximal end;
an absorbent body positioned at the distal end of the sheath; and
a hermetically closable pad positioned at the proximal end of the sheath.

2. The device according to claim 1, wherein the pad comprises a first surface facing, in use, the cavity, said first surface comprising an adhesive layer.

3. The device according to claim 1, wherein the pad comprises a line of weakness about which the pad can be folded.

4. The device according to claim 2, wherein the adhesive is a pressure-sensitive adhesive.

5. The device according to claim 2, wherein the adhesive is a reseal adhesive.

6. The device according to claim 2, wherein the adhesive layer is covered by a removable film.

7. The device according to claim 6, wherein the surface of the removable film facing, is use, the cavity, comprises an absorbent layer.

8. The device according to claim 2, wherein the pad comprises a second surface facing, in use, away from the cavity, said second surface comprising an impermeable layer.

9. The device according to claim 1, wherein the absorbent body comprises an absorbent layer.

10. The device according to claim 1, further comprising a removal string coupled to the distal end of the sheath.

11. The use for collecting a bodily sample from an anatomical cavity, of a device according to claim 1.

12. The use according to claim 11, wherein the anatomical cavity in the vaginal cavity, the rectal cavity or the buccal cavity.

13. A method for collecting a bodily sample from an anatomical cavity comprising the steps of collecting a sample from said anatomical cavity using a device according to claim 1 and hermetically closing the proximal end of the sheath.
